(19) United States
(12) Patent Application Publication
(10) Pub. No.: US 2003/0233047 A1
(43) Pub. Date: Dec. 18, 2003

(54) Dual-purpose medical curing device

(76) Inventor: Shun-Pin Lai, Taipei (TW)

Correspondence Address: ROSENBERG, KLEIN & LEE, 3458 ELLICOTT CENTER DRIVE-SUITE 101, ELLICOTT CITY, MD, 21043, US

(21) Appl. No.: 10/173,390

(22) Filed: Jun. 18, 2002

(51) Int. Cl. A61B 5/02

(52) U.S. Cl. 600/490

(57) ABSTRACT

A dual-purpose medical curing device of this invention includes: a main casing; a control circuit capable of executing blood pressure measurement and acupuncture point stimulation disposed inside the main casing; a bladder connected to or embedded in the main casing, the bladder being electrically connected to the control circuit; and a plurality of electrodes electrically connected to the control circuit. The plurality of electrodes are attached to skin of a tester's arm over acupuncture point of pericardium channel and proximity through a conductive line or a wristlet, by doing this, the blood pressure measurement and acupuncture point stimulation can be done at the same time, thereby preventing heart disease.

### BACKGROUND OF THE INVENTION

1. Field of the Invention

The present invention relates to a dual-purpose medical curing device. More particularly, this invention relates to a blood pressure measurement device capable of stimulating acupuncture points of a user, thereby preventing heart disease.

2. Description of the Prior Art

As lifestyle changes rapidly, people live in modern environment are facing a great deal of stress everyday. Stress brings about a lot of bad habits like unhealthy eating, smoking and drinking that can all lead to heart disease. Heart disease, which is the leading cause of death in the stressed society, is any condition that causes heart to malfunction. When the words “heart disease” are used generically, what people are usually referring to is Coronary heart disease, which leads to heart attacks and angina, ultimately caused by Atherosclerosis. But there are a wide range of other diseases of the heart, for example, Congestive Heart Failure, Valvular Heart Disease—Diseases of the heart valves, Cardiac Arrhythmias—Irregular heartbeats, Diseases of the Pericardium (sac around the heart), Diseases of the Myocardium (heart muscle), Endocarditis (infection of a heart valve), Congenital Heart Disease—birth defects of the heart.

Researchers have been racing to seek approaches to avoiding heart disease. In eastern, one of these approaches is an acupuncture point caring apparatus that generates far infrared radiation, electromagnetic wave, or implementing direct massage to Nei Guan (Inner Pass) acupuncture point of one's arm. As known in the art, Nei Guan (Inner Pass) acupuncture point belongs to so-called acupuncture points of pericardium channel system. Referring to FIG. 9, according to the Chinese medicine, pericardium channel of hand jue-yin is one of the 12 primary channels (also known as meridians or jing-luo) of human body. The pericardium channel of hand jue-yin is a channel in relation to human heart system. It is believed that frequently massaging the acupuncture point of pericardium channel comes out positive effects on preventing or curing certain heart disease. However, the prior art acupuncture point caring apparatus as mentioned has a shortcoming that it cannot measure and monitor a patient's (or tester's) blood pressure at the same time while he or she is using this kind of medical device.

### SUMMARY OF THE INVENTION

It is therefore the primary objective of the claimed invention to provide a dual-purpose medical curing device. The dual-purpose medical curing device of this invention comprises: a main casing; a control circuit capable of executing blood pressure measurement and acupuncture point stimulation disposed inside the main casing; a bladder connected to or embedded in the main casing, the bladder being electrically connected to the control circuit; and a plurality of electrodes electrically connected to the control circuit. The plurality of electrodes are attached to skin of a tester's arm over acupuncture point of pericardium channel and proximity through a conductive line or a wristlet, by doing this, the blood pressure measurement and acupuncture point stimulation can be done at the same time, thereby preventing heart disease.

Another objective of the claimed invention is to provide a dual-purpose medical curing device that is capable of easing emotional tension and curing tachycardia, arrhythmia, blood pressure variation, dyspepsia.

Still another objective of the claimed invention is to provide a dual-purpose medical curing device that has different structures including arm-integrated type or arm-separated type, wrist-integrated type or wrist-separated type, finger-integrated type or finger-separated type.

These and other objectives of the claimed invention will no doubt become obvious to those of ordinary skill in the art after reading the following detailed description of the preferred embodiment, which is illustrated in the various figures and drawings.

### BRIEF DESCRIPTION OF THE DRAWINGS

The accompanying drawings are included to provide a further understanding of the invention, and are incorporated in and constitute a part of this specification. The drawings illustrate embodiments of the invention and, together with the description, serve to explain the principles of the invention. In the drawings,

FIG. 1 is a circuit block diagram according to the present invention.

FIG. 2 is a circuit diagram according to the present invention.

FIG. 3 is a typical view according to the first preferred embodiment of the present invention.

FIG. 4 is a typical view according to the second preferred embodiment of the present invention.

FIG. 5 is a typical view according to the third preferred embodiment of the present invention.

FIG. 6 is a typical view according to the fourth preferred embodiment of the present invention.

FIG. 7 is a circuit diagram according to another embodiment of the present invention.

FIG. 8 is a typical view according to the fifth preferred embodiment of the present invention.

FIG. 9 is a schematic diagram illustrating acupuncture points of pericardium channel system according to Chinese medicine.

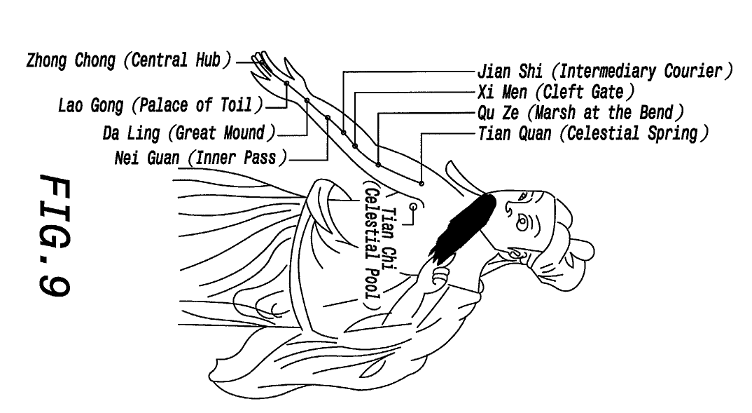

### DETAILED DESCRIPTION OF THE PREFERRED EMBODIMENTS

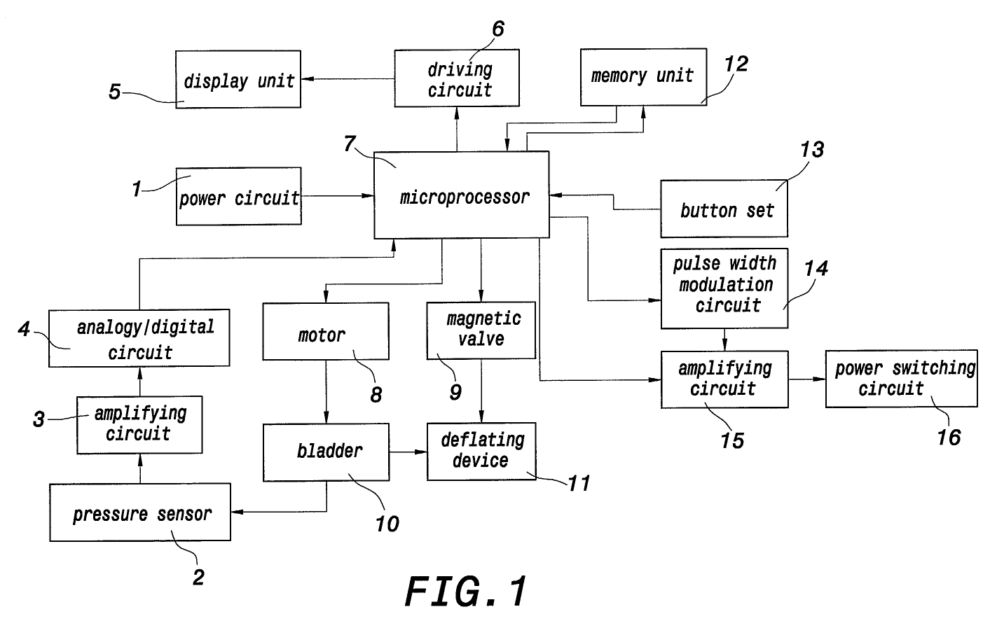

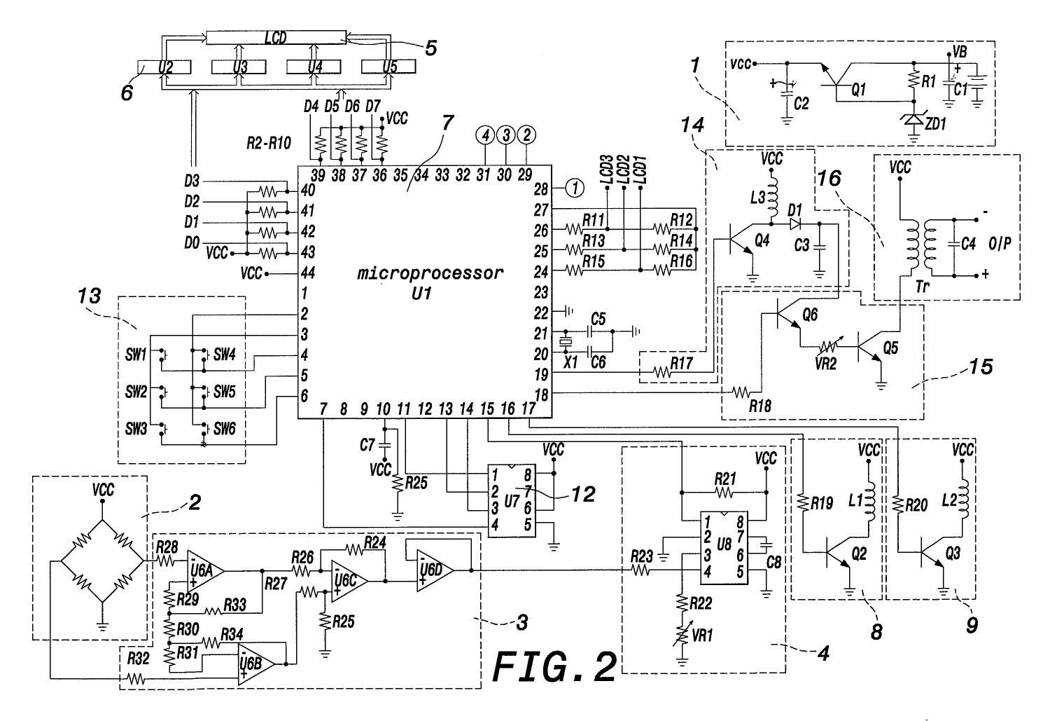

Referring to FIG. 1 to FIG. 3, this invention provides a dual-purpose medical device. As shown in figures, a main casing 20 is provided. Inside the casing 20 there is arranged a control circuit as illustrated in FIG. 2. The control circuit simultaneously executes blood pressure measurement and acupuncture point caring. More specifically, the control circuit is a low frequency electric stimulation circuit or a mid-frequency stimulation circuit.

As shown in FIG. 1, the control circuit comprises a power circuit 1, a pressure sensor 2, an amplifying circuit 3, a analogy/digital transform circuit 4, a display unit 5, a driving circuit 6, a microprocessor 7, a memory unit 12, button set 13, a pulse width modulation circuit 14, an amplifying circuit 15, and a power switching circuit 16.

As seen in FIG. 2, the power circuit 1 is comprised of a transistor Q1, resist R1, diode ZD1, capacitors C1 and C2, and battery BT. The power circuit 1 provides the circuit unit with proper work voltage.

The pressure sensor 2 is used to sense pressure inside a bladder or cuff 10.

The amplifying circuit 3 is comprised of amplifiers U6A to U6D, resists R24 to R32. The amplifying circuit 3 is electrically connected to the pressure sensor 2. The signals passed from the pressure sensor 2 are amplified by the amplifying circuit 3.

The analogy/digital transform circuit 4 is comprised of ICU8, capacitor C8, resists R21 to R23, VR1. The analogy/digital transform circuit 4 is electrically connected to output terminal of the amplifying circuit 3. The analogy/digital transform circuit 4 is used to transfer the amplified signals from the amplifying circuit 3 into digital signals.

The display unit 5 is a liquid crystal display (LCD) used to display sensed blood pressure reading.

The driving circuit 6 is comprised of driver ICs U2 to U5 and resists R2 to R16. The driving circuit 6 is electrically connected to the display unit 5 for driving the display unit 5.

The microprocessor 7 is comprised of control IC U1, quartz crystal X1, capacitor C5, C6, C7, and resist R25. The microprocessor 7 is electrically connected to power circuit 1, analogy/digital transform circuit 4, and driving circuit 6. The input port of the microprocessor 7 is connected with a motor 8 and a magnetic valve 9. The motor 8 is used to pump air into the bladder 10 through a conduit. A deflating device 11 is provided between the bladder 10 and the magnetic valve 9. The microprocessor 7 is operated according to input signals.

The memory 12 is comprised of a memory IC U7, which is electrically connected to the microprocessor 7 for accessing or storing data.

The button set 13 is comprised of switching devices SWI to SW6. The button set 13 is connected with the microprocessor 7.

The pulse width modulation circuit 14 is comprised of transistor Q4, diode Dl, capacitor C3, inductor L3, and resist R17. The pulse width modulation circuit 14 is electrically connected to output port of the microprocessor 7 for adjusting output strength of the electric stimulation circuit.

The amplifying circuit 15 is comprised of transistors Q5, Q6, resist R18, VR2. The amplifying circuit 15 is connected with output ports of the microprocessor 7 and the pulse width modulation circuit 14. The amplifying circuit 15 is controlled by the microprocessor 7 and is used to amplify output signals from the pulse width modulation circuit 14.

The power switching circuit 16 is comprised of coil Tr and capacitor C4. The power switching circuit 16 is electrically connected to output end of the amplifying circuit 15. The power switching circuit 16 is used to transform the voltage signals from the amplifying circuit 15 into high voltage signals.

Electrode 17 is attached to skin of one's arm over the acupuncture point of pericardium channel and proximity. The electrode 17 is electrically connected with the power switching circuit 16 and is powered by the power switching circuit 16 thereby providing the acupuncture point of pericardium channel with a proper stimulation current.

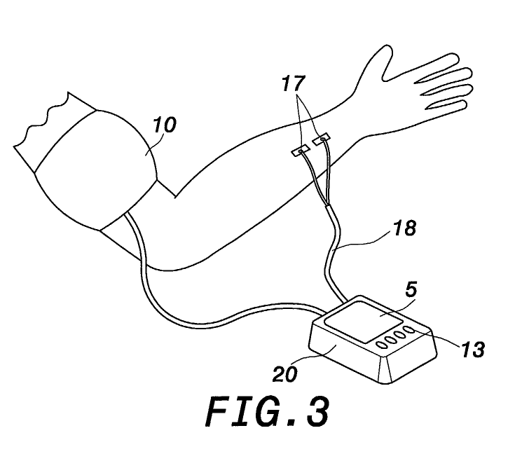

Referring to FIG. 3, according to the first preferred embodiment of the present invention, the bladder 10 is banded around a tester's arm. Electrodes 17 are attached to skin of the tester's arm over the acupuncture point of pericardium channel. Electrodes 17 are electrically connected to conductive line 18. By doing this, the blood pressure measurement and acupuncture point stimulation can be done at the same time. Or, the blood pressure measurement and acupuncture point stimulation can be done separately.

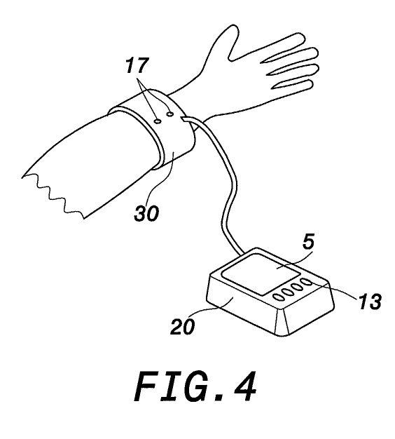

Referring to FIG. 4, according to the second preferred embodiment of the present invention, the electrodes 17 are embedded in the bladder wristlet 30. More specifically, the electrodes 17 are disposed on the interior surface of the bladder 30 such that when the bladder wristlet 30 is banded around a tester's arm or wrist, the electrodes 17 are able to contact the tester's skin. By doing this, the blood pressure measurement and acupuncture point stimulation can be done at the same time. Or, the blood pressure measurement and acupuncture point stimulation can be done separately.

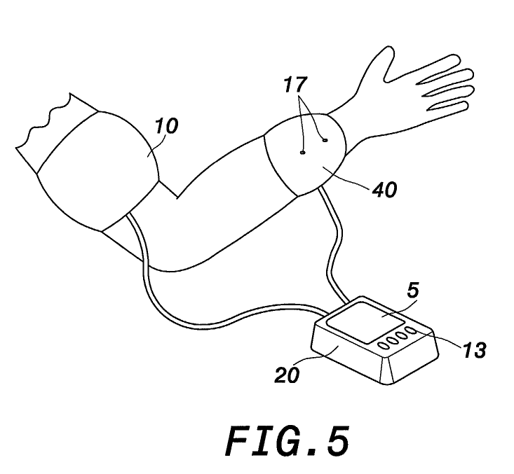

Referring to FIG. 5, according to the third preferred embodiment of the present invention, a wristlet 40 incorporating with several electrodes 17 is connected to the main casing 20. In use, the wristlet 40 is banded around a tester's arm near his or her wrist. The electrodes 17 embedded in the wristlet 40 are in contact with skin of the tester's arm. A bladder 10 is banded around the tester's upper arm as illustrated. By doing this, the blood pressure measurement and acupuncture point stimulation can be done at the same time. Or, the blood pressure measurement and acupuncture point stimulation can be done separately.

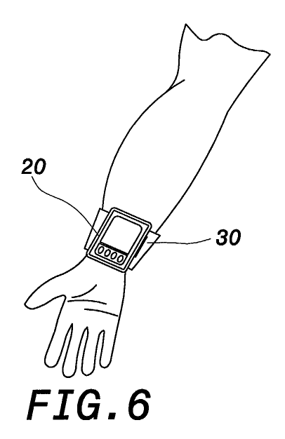

Referring to FIG. 6, according to the fourth preferred embodiment of the present invention, the main casing 20 is attached to the wristlet 30. The wristlet 30 incorporates with several electrodes on the interior surface of the wristlet 30. In use, the wristlet 30 is banded around a tester's arm near his or her wrist. The electrodes 17 embedded in the wristlet 40 are in contact with acupuncture point of pericardium channel of the tester's arm. By doing this, the blood pressure measurement and acupuncture point stimulation can be done at the same time. Or, the blood pressure measurement and acupuncture point stimulation can be done separately.

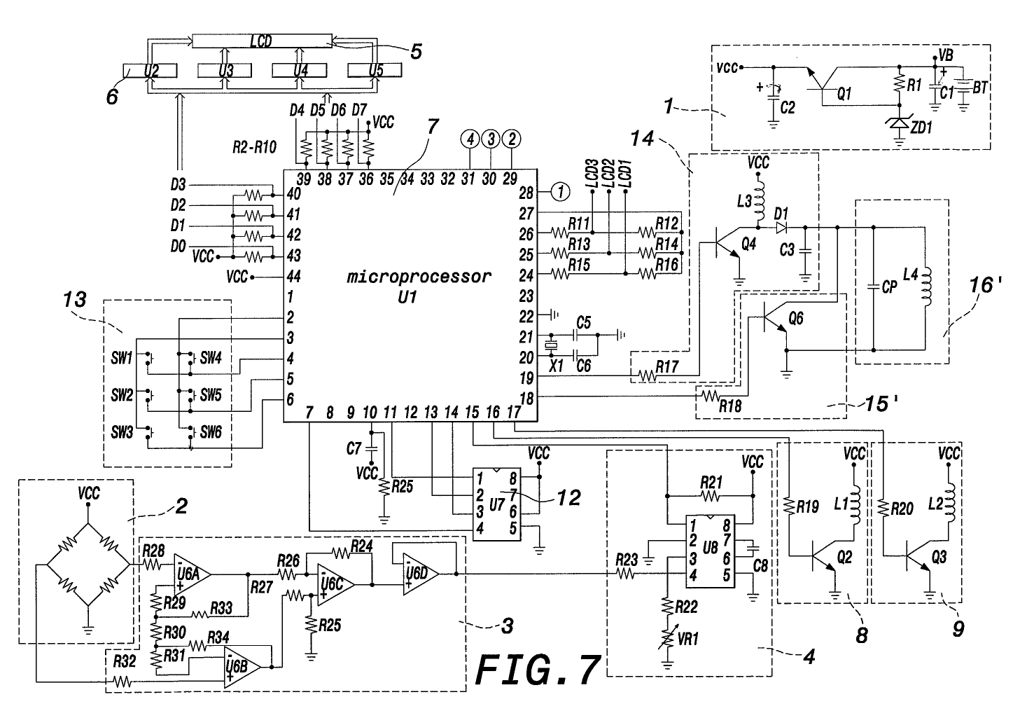

Referring to FIG. 7, the control circuit may be an electromagnetic wave stimulation circuit. As illustrated, the amplifying circuit 15′ is comprised of a transistor Q6, resist R18. The power switching circuit 16′ is comprised of coil L4 and capacitor CP.

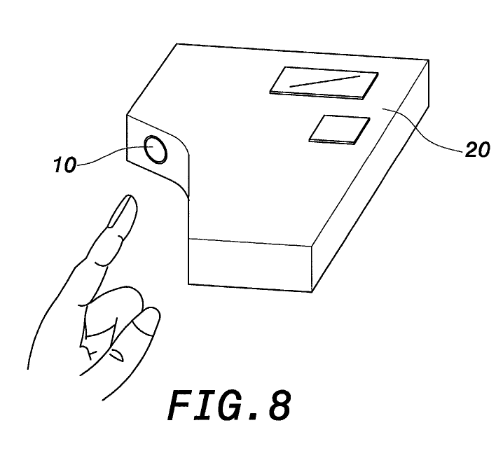

Referring to FIG. 8, the bladder 10 may be stored inside the main casing 20. In use, a tester inserts his finger into the bladder 10 to measure his blood pressure. At the same time, electrodes (not shown in this figure) are attached to skin of the tester's arm over the acupuncture point of pericardium channel. The electrodes are electrically connected to a conductive line. By doing this, the blood pressure measurement and acupuncture point stimulation can be done at the same time. Or, the blood pressure measurement and acupuncture point stimulation can be done separately.

To sum up, this invention presents several advantages as follows:

1. Heart disease can be prevented or curing by applying therapy by aid of the stimulation function of this invention.

2. Blood pressure can be monitored at the same time.

3. By stimulating acupuncture point of pericardium channel with a proper stimulation current, tachycardia, arrhythmia, blood pressure variation, dyspepsia can be cured.

4. There are various user-friendly structures available.

They are arm-integrated type or arm-separated type, wrist-integrated type or wrist-separated type, finger-integrated type or finger-separated type.

Those skilled in the art will readily observe that numerous modifications and alterations of the device may be made while retaining the teachings of the invention. For example, the RAM and the flash memory may be replaced with other similar memory devices known in the art. Accordingly, the above disclosure should be construed as limited only by the metes and bounds of the appended claims.

What is claimed is:

1. A dual-purpose medical curing device, comprising:
a main casing;
a control circuit capable of executing blood pressure measurement and acupuncture point stimulation disposed inside the main casing;
a bladder connected to or embedded in the main casing, the bladder being electrically connected to the control circuit; and
a plurality of electrodes electrically connected to the control circuit;
wherein the plurality of electrodes are attached to skin of a tester's arm over acupuncture point of pericardium channel and proximity through a conductive line or a wristlet, by doing this, the blood pressure measurement and acupuncture point stimulation can be done at the same time, thereby preventing heart disease.

2. The dual-purpose medical curing device of claim 1 wherein the main casing and the bladder are fabricated in structures of arm-integrated type or arm-separated type, wrist-integrated type or wrist-separated type, finger-integrated type or finger-separated type.

3. The dual-purpose medical curing device of claim 1 wherein the control circuit is a low-frequency electric stimulation circuit, a mid-frequency electric stimulation circuit, or an electromagnetic stimulation circuit.
